(19) United States
(12) Patent Application Publication
(10) Pub. No.: US 2023/0226300 A1
(43) Pub. Date: Jul. 20, 2023

(54) CAPNOGRAPHY FACEMASK

(71) Applicant: Chris Salvino, SCOTTSDALE, AZ (US)

(72) Inventor: Chris Salvino, SCOTTSDALE, AZ (US)

(21) Appl. No.: 18/096,331

(22) Filed: Jan. 12, 2023

Related U.S. Application Data
(60) Provisional application No. 63/300,486, filed on Jan. 18, 2022.

(51) Int. Cl. A61M 16/06; A61B 5/083; A61M 16/08

(52) U.S. Cl. CPC A61M 16/0672; A61B 5/0836; A61M 16/085; A61M 16/0683

(57) ABSTRACT

An oxygen facemask that has pathogen filtering capability can be used in conjunction with a capnometer to measure carbon dioxide levels in a patient. Facemask can be used to provide critical breathing assistance to patients. In certain instances, a patient’s blood CO2 level can be the difference between a good versus a bad outcome. Accordingly, in certain circumstances there is great value in evaluating a patient’s CO2 with a capnometer from breath exiting the facemask, which correlates to CO2 in their blood. Because some of these patients carry airborne transmissible viruses, an exhalent filtration system is envisioned included in the facemask that filters the patient’s breath from those helping the patient in a common space. The exhalent filtration system incorporates both filtering air exiting the facemask directly into a common space and indirectly into the common space by way of the capnometer.

### CROSS-REFERENCE TO RELATED APPLICATIONS

This application claims priority to and the benefit of U.S. Provisional Pat. Application No. 63/300,486 entitled CAPNOGRAPHY FACEMASK, filed on Jan. 18, 2022.

### FIELD OF THE INVENTION

The present embodiments are directed to capnography oxygen facemask.

### BACKGROUND OF THE INVENTION

Oxygen facemasks, such as non-rebreather (NRB) facemasks and regular oxygen facemasks (RFM), are used in medical emergencies where people in distress require oxygen therapy. Such emergencies include physical trauma, chronic airway limitations, chronic obstructive pulmonary diseases, smoke inhalation, carbon monoxide poisoning and other respiratory impairments.

A typical oxygen facemask covers both the nose and the mouth of a patient. It is constrained to their face by way of an elastic strap that goes around the back of the patient’s head. The elastic strap is essentially attached to the perimeter of the typical facemask (within a quarter/half an inch from the perimeter). It is to innovations related to this subject matter that the embodiments of the invention is generally directed.

### SUMMARY OF THE INVENTION

The present embodiments are directed to capnography facemasks with some embodiments directed to filtrating exhalant that could be contaminated with airborne biological pathogens from a patient wearing/using a capnography facemask. Hence, several different capnography facemask embodiments are presented herein including an oxygen facemask that has pathogen filtering capability, which can be used in conjunction with a capnometer to measure carbon dioxide levels in a patient’s breath. Because, a patient’s blood carbon dioxide (CO2) level can be the difference between a good and a bad outcome, there is value in measuring a patient’s breath CO2 level. Some of the embodiments herein are directed to measuring CO2 levels of a patient’s breath after exiting a facemask with a capnometer. The CO2 levels in the patient’s breath correlate with blood CO2 levels. To address exhaling airborne transmissible viruses, an exhalent filtration system is disclosed which is included in the facemask. The exhalent filtration system filters the patient’s breath, which protects those helping the patient. The exhalent filtration system incorporates both filtering air exiting 1) the facemask and 2) the capnometer.

With this in mind, certain embodiments of the present invention therefore contemplate a capnography facemask generally comprising a facemask cup that is connected to a capnometer by way of an exhaled air filter. The facemask cup possesses an outer rim defined by a nose bridge rim region configured to traverse a wearer’s nose bridge, two cheek rim regions configured to traverse a wearer’s cheeks, and a chin rim region configured to traverse a wearer’s face below their mouth, such as along their jawline, for example. The face covering cup defines a nose region that is configured to cover the wearer’s nose and a mouth region configured to cover the wearer’s mouth. A facemask oxygen inlet tube outwardly extends from the facemask cup in the nose region. The capnography the facemask is connected to an exhaled air filter located at the mouth region, the exhaled air filter comprising at least one primary exit aperture and a sample exit port. The capnography the facemask is configured to be worn on a wearer’s face when being used, there is nothing between the at least one primary exit aperture and an exterior environment when the capnography the facemask is being used. There is a capnometer between the sample exit port in the exterior environment when the capnography the facemask is being used.

Yet another embodiment of the present invention contemplates a capnography facemask assembly comprising a the facemask including: 1) a face covering cup defining a cup exterior and a cup interior, the face covering cup terminates at a cup rim, 2) the facemask further comprising a facemask oxygen inlet tube located at a cup nose region and extending outwardly from the cup exterior, and 3) an exhale port located at a cup mouth region, the facemask configured to cover a mouth and nostrils of a person, which essentially seals against a face of a person at an interface between the face and the cup rim. The cup interior faces the person’s face and the cup exterior faces an exterior environment when worn on the person’s face. A capnometer comprises a capnometer intake port and a capnometer exit port. An exhaled air filter is disposed between the capnometer and the exhale port. The exhaled air filter comprises filter media, at least one primary exit aperture, and a sample exit port. The exterior environment is in communication with the cup interior via the at least one primary exit aperture and via the capnometer exit port.

Still, another embodiment envisions a method for using a capnography facemask assembly, the method comprising providing a facemask that includes a face covering cup, a capnometer, and an exhaled air filter. The face covering cup terminates at a cup rim, which defines a cup exterior and a cup interior. The capnometer has a capnometer intake port and capnometer exit port. An exhaled air filter is interposed between the capnometer and an exhale port located at a cup mouth region of the face covering cup. The method continues with covering a mouth, nostrils and a portion of a person’s face with the facemask with the cup interior facing the person’s face. The cup rim essentially seals against the person’s face. Volume between the cup interior and the person’s face defines an interior environment. The capnometer intake port is attached to a filter sample port of the exhaled air filter via a sample exhaled air tube. Essentially all air that exits from the interior environment is filtered to an exterior environment via the exhaled air filter (filtered air). All of the filtered air exits into the exterior environment via A) the capnometer exit port and B) at least one primary exit aperture of the exhaled air filter.

### BRIEF DESCRIPTION OF THE DRAWINGS

FIG. 1 is an isometric line drawing of a capnography oxygen facemask assembly embodiment consistent with embodiments of the present invention;

FIG. 2 is an isometric line drawing of the facemask embodiment 110 consistent with optional facemask embodiments conceivable within the scope and spirit of the present invention;

FIG. 3 illustratively depicts a line drawing of an exploded isometric view of the facemask assembly 100 without the capnometer 160 consistent with embodiments of the present invention;

FIG. 4 is an isometric exploded view line drawing of an exhaled air filter consistent with embodiments of the present invention;

FIG. 5 is a front view line drawing of the facemask assembly 110 consistent with embodiments of the present invention. In this image, the different regions of the face covering cup rim 212 are depicted as spanning the double-sided arrows;

FIG. 6 is a side view line drawing of the facemask assembly 100 consistent with embodiments of the present invention; and

FIG. 7 is a rear view line drawing of the facemask assembly 100 consistent with embodiments of the present invention.

### DETAILED DESCRIPTION

Initially, this disclosure is by way of example only, not by limitation. Thus, although the instrumentalities described herein are for the convenience of explanation, shown and described with respect to exemplary embodiments, it will be appreciated that the principles herein may be applied equally in other similar configurations involving the subject matter directed to the field of the invention. The phrases “in one embodiment”, “according to one embodiment”, and the like, generally mean the particular feature, structure, or characteristic following the phrase, is included in at least one embodiment of the present invention and may be included in more than one embodiment of the present invention. Importantly, such phases do not necessarily refer to the same embodiment. If the specification states a component or feature “may”, “can”, “could”, or “might” be included or have a characteristic, that particular component or feature is not required to be included or have the characteristic. As used herein, the terms “having”, “have”, “including” and “include” are considered open language and are synonymous with the term “comprising”. Furthermore, as used herein, the term “essentially” is meant to stress that a characteristic of something is to be interpreted within acceptable tolerance margins known to those skilled in the art in keeping with typical normal world tolerance, which is analogous with “more or less.” For example, essentially flat, essentially straight, essentially on time, etc. all indicate that these characteristics are not capable of being perfect within the sense of their limits. Accordingly, if there is no specific +/- value assigned to “essentially”, then assume essentially means to be within +/- 2.5% of exact. The term “connected to” as used herein is to be interpreted as a first element physically linked or attached to a second element and not as a “means for attaching” as in a “means plus function”. In fact, unless a term expressly uses “means for” followed by the gerund form of a verb, that term shall not be interpreted under 35 U.S.C. § 112(f). In what follows, similar or identical structures may be identified using identical callouts.

With respect to the drawings, it is noted that the figures are not necessarily drawn to scale and are diagrammatic in nature to illustrate features of interest. Descriptive terminology such as, for example, upper/lower, top/bottom, horizontal/vertical, left/right and the like, may be adopted with respect to the various views or conventions provided in the figures as generally understood by an onlooker for purposes of enhancing the reader’s understanding and is in no way intended to be limiting. All embodiments described herein are submitted to be operational irrespective of any overall physical orientation unless specifically described otherwise, such as elements that rely on gravity to operate, for example.

The present embodiments are directed to capnography oxygen facemasks with some embodiments directed to filtrating exhalant. Capnography is a non-invasive measurement of the partial pressure of CO2 from a person’s airway during inspiration and expiration. It provides physiologic information on ventilation, perfusion, and metabolism, which is important for airway management. Capnography measurements are obtained with a capnometer, which is a monitoring device that measures and numerically displays the concentration of CO2 in exhalent (exhaled air/gas). Capnography works by measuring the amount of infrared light that is absorbed by CO2. Exhaled air passes down a tube (that is connected to a capnometer), through the capnometer and into ambient air. Because capnometers are used in emergency rooms, hospital rooms, or other general healthcare rooms, the exhaled air ultimately passes from the capnometer into these rooms. As exhaled gas passes through the capnometer, a beam of infrared light shines on an infrared sensor by way of passing through the exhaled air.. The amount of infrared light that reaches the infrared sensor corresponds to the amount of CO2 in the patient’s exhaled gas.

Though capnometers can comprise general internal filtration capability (typically within the capnometer housing) or at the capnometer, such general filtration capability is for dust greater than 5 microns. In other words, a capnometer’s general internal filtration capability is to protect the instrumentation from getting damaged by dust particles. Hence, filtering out viruses or bacteria provides no benefit to the functionality of a capnometer and therefore has never been integrated in a capnometer. Certain aspects of the present invention are directed to a filter system disposed at a facemask or in a tube connecting a facemask to a capnometer. Certain embodiments envision this filter being disposed in a location between the facemask in the capnometer instead of being at or in a capnometer, thereby making it much easier to access and change or otherwise replace the filter with a new one. Certain other embodiments envision including a biological filter system at the capnometer that filters biological particles, such as bacteria and/or viruses. Certain other aspects of the present invention are directed to filtering viruses from exhalant thereby reducing exposure of a potentially dangerous virus to those working with the patient in the same room. The exhalent filtration system incorporates both filtering air exiting the facemask directly into a common space and indirectly into the common space by way of the capnometer. Additionally, by filtering the exhaled breath before it reaches the capnometer, the capnometer will not get contaminated by a patient infected with a virus or bacterial infection that spreads through breathing.

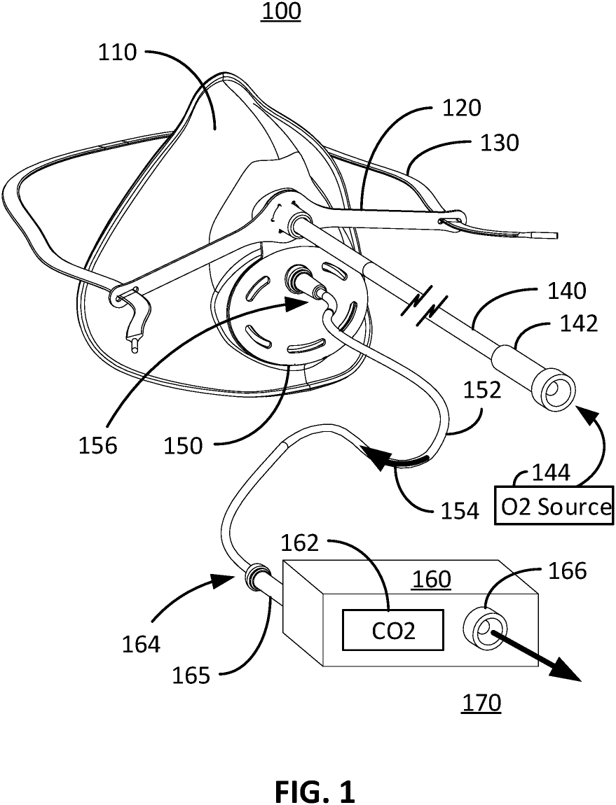

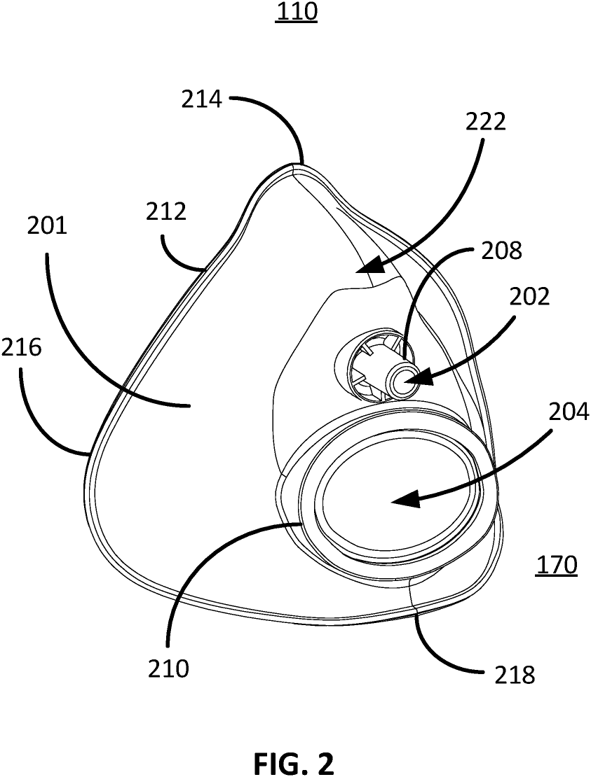

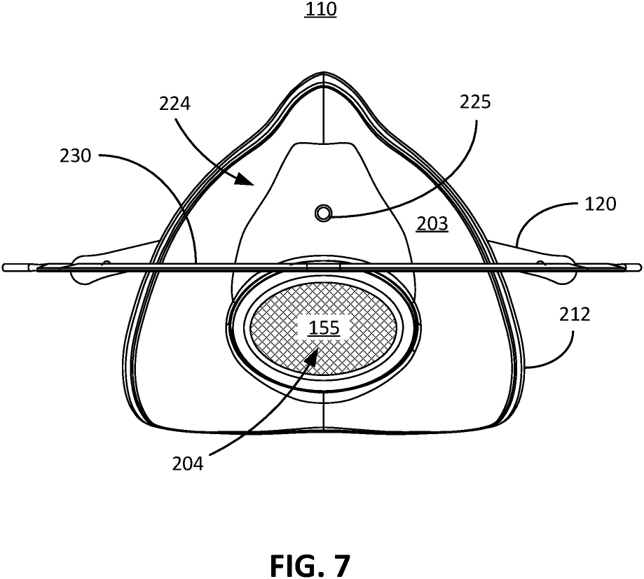

FIG. 1 is an isometric line drawing of a capnography oxygen facemask assembly embodiment consistent with embodiments of the present invention. For purposes of brevity, the capnography oxygen facemask 110 is typically referred to herein as “facemask”. The primary components of the facemask assembly 100 generally includes a facemask 110, a front facemask retention brace 120 that cooperates with a facemask retention strap 130, and oxygen inlet tube 140, an exhaled air filter 150, a sample exhaled air tube 152, and a capnometer 160. The facemask 110 is configured and adapted to fit snugly around a person’s face (not shown) covering their nose and mouth when worn (i.e., the wearer’s face). The facemask 110 is secured to the wearer’s face by way of the facemask retention strap 130 that goes around the back of the wearer’s head. The facemask retention strap 130 cooperates with the front facemask retention brace 120, which pulls the flexible facemask 110 snugly towards the wearer’s face. Oxygen is supplied to the facemask 110 via the oxygen inlet tube 140 via an oxygen source 144. In the present embodiment, the oxygen inlet tube 140 comprises an oxygen source connector 142 configured and adapted to connect to an oxygen source 144, such as an oxygen tank or oxygen supply line (not shown). The exhaled filter 150 is located in front of the wearer’s mouth and provides filtration of exhaled air from the wearer the is potentially exhaled into a room of medical workers. A sampled exhaled air tube 152 is attached to the exhaled air filter 150 at the air tube filter end 156 and the capnometer intake port 165, of the capnometer 160, at the air tube capnometer end 164. The sampled exhaled air tube 152 is configured and adapted to carry filtered exhaled air 154 to the capnometer 160. The capnometer 160 measures the carbon dioxide (CO2) in the filtered exhaled air and displays the result on the capnometer display screen 162. The post capnometer filtered exhaled air 154 is expelled into the ambient/exterior environment 170 via a capnometer exit port 166. As should be appreciated, certain embodiments of the facemask assembly 100 are envisioned to be used to provide critical breathing assistance to patients. The capnometer 160 is configured to assess the patient’s CO2 level to alert caretakes if CO2 blood levels become dangerous. The filters help prevent airborne transmissible viruses or other pathogens that may be present in a patient from contaminating the common space frequented by medical personnel. FIG. 2 is an isometric line drawing of the facemask embodiment 110 consistent with optional facemask embodiments of the present invention. The facemask 110 generally comprises a face covering cup 201 that is configured to cover both a human mouth and human nostrils (not shown). In this embodiment, the face covering cup 201 is a unitary flexible molded PVC cup, which could be clear or opaque. A facemask oxygen inlet tube 208, comprising a facemask oxygen inlet port 202, extends outwardly from the face covering cup front 222. In this embodiment, the facemask oxygen inlet tube 208 is configured to connect with an oxygen inlet tube 140 of FIG. 1. The facemask interior 203 of FIG. 7 is in communication with the oxygen source 144 via the oxygen inlet port 202 and the oxygen inlet tube 140. The face covering cup rim 212 extends over the bridge of a wearer’s nose at a nose bridge rim region 214, over a wearer’s cheeks at a cheek rim region 216 and below a person’s mouth or optionally over a wearer’s chin, or jawline, at a chin rim region 218. The face covering cup rim 212 is configured to essentially seal against the wearer’s face when being pulled tightly against the wearer’s face via the facemask retention strap 130 and retention brace 120 of FIG. 1. The facemask oxygen inlet tube 208 is positioned at or near a wearer’s nostrils. An exhaled air filter rim 210 is configured to matingly cooperate with the exhaled air filter 150 of FIG. 1. Examples of the air filter rim 210 matingly cooperating with the exhaled air filter 150 can be to snap or otherwise connect the exhaled air filter 150 to cover the exhale port 204. The facemask interior 203 and the exterior environment 170 are in communication via the exhale port 204.

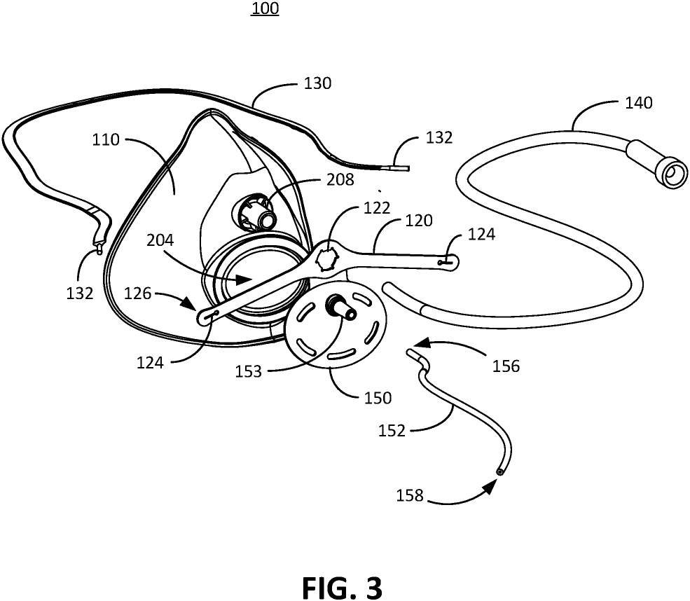

FIG. 3 illustratively depicts a line drawing of an exploded view of the facemask assembly 100 without the capnometer 160 consistent with embodiments of the present invention. Certain embodiments contemplate each of the exploded elements being removably attached in a way that provides substitution of like elements. With respect to the facemask retention strap 130, certain embodiments envision this being an elastic strap while other embodiments envision a non-elastic strap much like a shoelace. The retention strap ends 132 engage or otherwise thread through the retention brace slots 124 located at the retention brace ends 126 of the retention brace 120. The retention brace slots 124 retain, or otherwise lock, the facemask retention strap 132 the retention brace 120 at the retention brace ends 126. The retention brace 120 slidingly engages the facemask oxygen inlet tube 208 by way of a centrally located retention brace connecting hole/aperture 122. When worn by a wearer/person, the retention brace 120 pulls the facemask onto the front of the wearer’s face because the retention brace 120 is located between a person’s nostrils and upper lip. The retention brace 120 reduces the “dead space” of the facemask 110 to the wearer’s face. Dead space is the volume between a wearer’s face and the facemask interior 203. Hence, the greater the volume between the wearer’s face and the facemask interior 203, the greater the average distance between the facemask interior 203 and the wearer’s face.

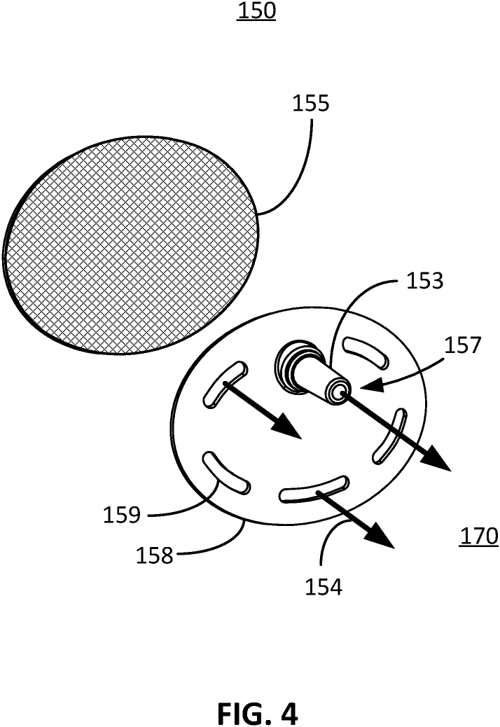

In the present embodiment, the oxygen inlet tube 140 is held in place in the interior of the facemask oxygen inlet tube 208 by way of a friction fit. Other ways of connecting the oxygen inlet tube 140 to the facemask oxygen inlet tube 208 are well known in the mechanical arts. The exhaled air filter 150 is sized to completely cover the exhale port 204, as shown, and as will be discussed in further detail in conjunction with FIG. 4. Importantly, the exhaled air filter 150 is disposed between the facemask 110 and the capnometer 160, of FIG. 1, but at or near the facemask 110 (within a couple of inches of the facemask 110). This is important because the exhaled air filter 150 also serves to filter a person’s breath exiting the facemask 110, which does not go through the capnometer 160. And, in the present embodiment, the sample exhaled air tube 152 is friction fit inside of the filter’s sample exhaled air tube 153. However, other connecting options can be used without deviating from the scope and spirit of the present invention, which are known to those skilled in the mechanical arts.

FIG. 4 is an isometric exploded view line drawing of an exhaled air filter consistent with embodiments of the present invention. Certain embodiments envision the filter media 155 being made from a virus filtering material, such as N-95 or N-99 filter material, or some other filter material that is efficient in filtering viruses and/or bacteria (germs) from passing therethrough. An N-95 filter media comprises a pore size distribution where 95% of the pores are less than 0.3 µm. In certain embodiments, the filter media 155 is bonded or adhered to the filter plate 158, which can be rigid or semi-rigid plastic plate permeated with holes or slots. Certain embodiments contemplate that the same filter media 155 filters a patient’s breath 154 for both the breath 154 that exits from the facemask 110 directly into the exterior environment 170 (out the exit slots 159) and enters/exits the capnometer 160. The filter plate 158 comprises a plurality of exit slots 159 through which filtered air 154 is breathed out of the facemask 110 and into the exterior environment 170. A portion of the filtered air 154 is directed through the exhaled air sample port 157, at the distal end of the filter’s sample exhaled air tube 153, where the filtered air 154 passes through the capnometer 160 of FIG. 1. Though there are five exit slots 159, there could be greater or fewer perforations with potentially different shapes in the filter plate 158 that lead to the exterior environment 170. An exit slot 159 is an embodiment of a primary exit aperture, which provides a pathway for exhaled air to be filtered when it is expelled directly into a room or some other ambient environment 170. The primary aperture 159 is defined as an opening through which filtered exhaled air 154 gets breathed out into an ambient environment 170 without anything between the primary aperture 159 and the ambient environment 170. Importantly, the exhaled air filter 150 is disposed between the facemask 110 and the capnometer 160, of FIG. 1, but at or near the facemask 110, such as within 2 inches of the facemask 110. This is important because the exhaled air filter 150 also serves to filter breath exiting the facemask 110 that does not go through the capnometer 160. Accordingly, assuming that the facemask 110 is tightly sealed against a wearer’s face, the exhaled air filter 150 will filter all exhaled breath 154 from the wearer whether the exhaled breath 154 flows into the exterior environment directly after passing through the filter 150 or by way of the capnometer 160.

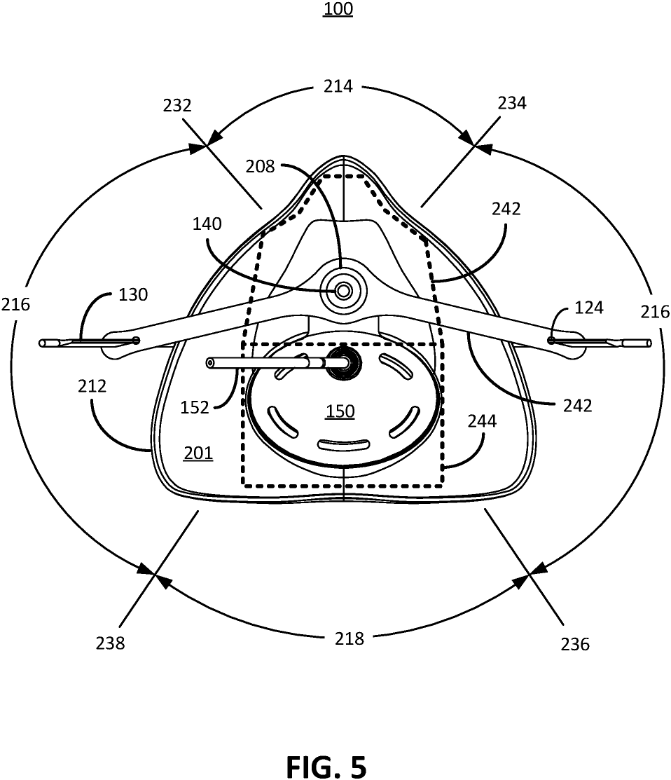

FIG. 5 is a front view line drawing of the facemask assembly 110 consistent with embodiments of the present invention. In this image, the different regions of the face covering cup rim 212 are depicted as spanning the double-sided curved arrows. Specifically, the nose bridge rim region 214 spans between rim demarcation lines 232 and 234, the cheek rim region 216 on the left side of the face covering cup 201 spans between rim demarcation lines 232 and 238, the cheek rim region 216 on the right side of the face covering cup 201 spans between rim demarcation lines 234 and 236, and the chin rim region 218 spans between rim demarcation lines 236 and 238. It should be appreciated that the different regions of the cup rim 212 are approximate, such as within 10% of the periphery of the cup rim 212. Both the sample exhaled air tube 152 and the oxygen inlet tube 140 are pointing out of the page. For reference, the exhaled air filter 150 and the retention brace 120 are shown in-line with the page. The facemask retention strap 130 is also shown sticking through or otherwise cooperating with the retention brace slots 124. The facemask oxygen inlet tube 208 is located in the nose region 242, which is depicted by the dotted area 242. The exhaled air filter 150 is located in the mouth region 244, which is depicted by the dotted rectangle 244.

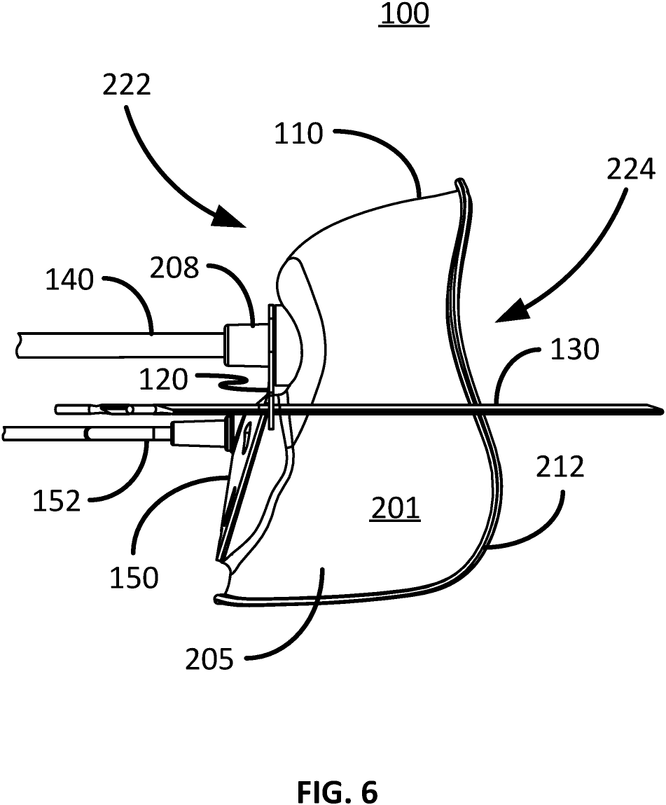

FIG. 6 is a side view line drawing of the facemask assembly 100 consistent with embodiments of the present invention. This image shows the exhaled air filter 150 between the facemask 110 and the sample exhaled air tube 152. In certain embodiments, the exhaled air filter 150 is not in contact with the face covering cup 201 but rather spaced in front of the facemask covering cup 201. The oxygen inlet tube 140 is depicted extending to the left of the retention brace 120, which is connected to the facemask retention strap 130. The facemask front 222 is essentially where the exhaled air filter 150 and the facemask oxygen inlet port 208 reside while the face covering cup rim 212 generally defines the facemask back 224. For reference, the facemask cup exterior surface 205 is presented in this figure (as well as FIGS. 1-3 and 5).

FIG. 7 is a rear view line drawing of the facemask assembly 100 consistent with embodiments of the present invention. This depiction is of the facemask back 224 with the face covering cup rim 212 extending prominently facing out of the page. This also shows the facemask retention strap 230 extending out of the page. This perspective shows the facemask interior 203 of the face covering cup 201. This perspective further shows the facemask inlet orifice 225 which is in communication with the facemask oxygen inlet port 202 of FIG. 2 and the exhaled port 204 leading into the filter media 155.

With the present description in mind, below are some examples of certain embodiments illustratively complementing some of the methods and apparatus embodiments to aid the reader. The elements called out below in view of the various figures are examples provided to assist in understanding the present invention and accordingly should not be considered limiting.

In that light, one embodiment of the present invention described in view of FIG. 1 envisions a capnography facemask 110 generally comprising a facemask cup 201 that is connected to a capnometer 160 by way of an exhaled air filter 150. The facemask cup 201 possesses an outer rim 212 defined by a nose bridge rim region 214 configured to traverse a wearer’s nose bridge, two cheek rim regions 216 configured to traverse a wearer’s cheeks, and a chin rim region 218 configured to traverse a wearer’s jawline, see FIG. 5. The wearer is a person wearing or otherwise using the capnography facemask 110 and is not shown in figures. The face covering cup 201 defines, among other things, a nose region 242 configured to cover the wearer’s nose and a mouth region 244 configured to cover the wearer’s mouth, again refer to the dotted lined areas of FIG. 5. As shown, facemask oxygen inlet tube 208 outwardly extending from the facemask cup 201 in the nose region 242. By outwardly extending, it is meant that the facemask oxygen inlet tube 208 is extending from the cup exterior surface 205, which is shown extending to the left in FIG. 6. The capnography facemask 110 is connected to an exhaled air filter 150 located at the mouth region 244, and specifically over the exhaled port 204 of FIG. 2. The exhaled air filter 150 comprises at least one primary exit aperture 159 and a sample exit port 157. The primary exit aperture 159 can be the plurality of exit slots depicted in FIG. 4, one large aperture, some other shape, etc. The primary exit aperture 159 is envisioned to generally support sufficient exiting of a patient’s exhaled breath to become filtered when leaving the facemask 110 to protect those around the patient from pathogens that may be in the patient’s breath. The capnography facemask 110 is configured to be worn on a wearer’s face when being used (i.e., warn and breathed through). There is nothing between the at least one primary exit aperture 159 and an exterior environment 170 when the capnography facemask 110 is being used, meaning the filtered exhaled air/breath 154 passes directly from the filter 150 without any intermediate physical element or device. On the other hand, there is a capnometer 160 between the sample exit port 157 in the exterior environment 170 when the capnography facemask 110 is being used, meaning the filtered exhaled air/breath 154 that goes through the sample exit port 157 does not directly exit into the environment but rather exits indirectly through the capnometer exit port 166.

Embodiments of the capnography facemask 110 are additionally envisioned to include the facemask oxygen inlet tube 208 linking to an oxygen source 144 by way of an oxygen inlet tube 140, as shown in FIG. 1.

The capnography facemask embodiment 110 is further envisioned to be wherein the outer rim 212 is configured to essentially seal against the wearer’s face when the capnography facemask 110 is being used. “Essentially seal” is meant to be within the standards of today’s Facemasks. It is envisioned that the capnography facemask 110 is pulled securely against the wearer’s face to pretty much seal against the wearer’s face, however in reality there may be small ‘unavoidable’ gaps between the outer rim 212 and the wearer’s face. The capnography facemask 110 is considered “essentially sealed” against a person’s face even with these small ‘unavoidable’ gaps.

The capnography facemask embodiment 110 further contemplates that when the capnography facemask 110 is being used essentially all air exiting the capnography facemask 110 is filtered air 154 before entering the exterior environment 170. In more plain language, when a person is wearing the capnography facemask 110 oxygen provided by the oxygen source 144 is directed towards the wearer’s nostrils but when the person exhales, essentially all of the exhaled breath is filtered by the exhaled air filter 150 (or more specifically the filter media 155) as it moves into the exterior environment 170. In this way, people in the vicinity of the person wearing the capnography facemask 110 are protected from any viruses actively shedding from the person. This can further include wherein a first portion of the filtered air 154 exits into the exterior environment 170 via the at least one primary exit aperture 159 and a second portion of the filtered air 154 exits into the exterior environment 170 via a capnometer exit port 166 of a capnometer 160. The capnometer 160 connected to the sample exit port 157 via a simple exhaled air tube 152. Accordingly, the majority of the exhaled breath ( > 51% filtered breath 154) goes through the exit slots 159 directly into the exterior environment 170 but a minority of the exhaled filtered breath 154 ( < 49%) is first sampled by the capnometer 160 for CO2 levels of the person wearing the capnography facemask 110 before it goes into the same exterior environment 170.

Certain embodiments contemplate the breathed out air 154 only going through the capnometer 160. In other words, the sample port 157 is envisioned to be sufficiently large enough to accommodate all of the exhaled air 154. Accordingly, in this embodiment, there is a biological filter system interposed between this facemask embodiment and the capnometer 160. Certain embodiments contemplate a filter system being placed by way of an intermediate tube between an oxygen facemask and the capnometer 160. Because the capnometer 160 provides the only exit port to the exterior environment 170 for the facemask arrangement (like arrangement 100, which includes all of the general elements albeit adjusted to suit the present embodiment), the only exit port in the facemask embodiment is to the capnometer 160.

The capnography facemask embodiment 110 further imagines the at least one primary exit aperture 159 being a slotted aperture. Of course, a person skilled in the art in possession of the technology disclosed herein will appreciate that the at least one primary exit aperture 159 can be about any shape and size that comports with the desired exit filtered air flow.

The capnography facemask embodiment 110 further contemplates comprising a front facemask retention brace 120 that defines a retention brace center 128 located between the nose region 242 and the mouth region 244. The front facemask retention brace 120 extends to retention brace distal ends 126 on either side of the retention brace center 128 toward the cheek rim regions 216, see FIGS. 1 and 3 for example. The retention brace 120 facilitates squeezing the flexible face covering cup 201 more closely to a person’s face thereby reducing the amount of dead space in the capnography facemask 110. Dead space is defined as the volume between the facemask interior surface 203 and the person’s face. This embodiment is envisioned to be actionable by way of a facemask retention strap 130 that connects to the facemask retention brace 120 essentially at the retention brace distal ends 126. One embodiment further envisions the front facemask retention brace 120 comprising a brace aperture 122 at the retention brace center 128, wherein the brace aperture 122 and circles the facemask oxygen inlet tube 208, as shown in FIGS. 1 and 3.

Yet another embodiment of the present invention explores a capnography facemask assembly 100 comprising that generally comprises a facemask 110 including: 1) a face covering cup 201 defining a cup exterior 205 and a cup interior 203, the face covering cup 201 terminates at a cup rim 212, 2) the facemask 110 further comprising a facemask oxygen inlet tube 208 located at a cup nose region 242 and extending outwardly from the cup exterior 205, and 3) an exhale port 204 located at a cup mouth region 244. The different major components (numerals 1-3) are not considered to be limiting of the major components unless otherwise stated but rather to help distinguish the elaborating language to each of the different major components. The facemask 110 is configured to cover a mouth and nostrils of a person and essentially seal against a face of a person at an interface between the face and the cup rim 212. The cup interior 203 facing the face and the cup exterior 205 facing an exterior environment 270 when worn on the face. A capnometer 160 comprising a capnometer intake port 165 and a capnometer exit port 166. An exhaled air filter 150 is disposed between the capnometer 160 and the exhale port 204. The exhaled air filter 150 comprising at least a filter media 155 (such as an N-95 or N-99 filter fabric), at least one primary exit aperture 159, and a sample exit port 157. The exterior environment 170 is in communication with the cup interior 203 via the at least one primary exit aperture 159 and via the capnometer exit port 166. Meaning, the exhaled air filter 150, which filters essentially all of the wearer’s breath as it exits the facemask 110 must either pass into the exterior environment 170 by way of the exit slots 159 or the capnometer 160. In this way, all breath exhaled from the wearer’s filtered whether it sampled through the capnometer or simply directly breathed out of the facemask 110 via the exit slots 159.

The capnography facemask assembly embodiment 100 as described above can further comprise an oxygen source 160 connected to the facemask oxygen inlet tube 208, the exterior environment 170 is only in communication with the cup interior 203 via the at least one primary exit aperture 159 and via the capnometer exit port 166.

The capnography facemask assembly embodiment 100 as described above can be wherein the exhaled air filter 150 is connected to the facemask 110 and covers the exhale port 204. Certain embodiments envision the exhaled air filter 150 not contacting the face covering cup 201 whatsoever but nonetheless covers the exhale port 204 by way of an intermediate member (such as a gasket, not shown). Because certain embodiments contemplate the exhaled air filter 150 being its own component, the exhaled air filter 150 can be removed from cooperating with a facemask 110, discarded and replaced with a new/unused exhaled air filter 150. Swapping out a used exhaled air filter 150 with a new exhaled air filter 150 can further protect those working with the wearer/patient in the same or otherwise common exterior environment 170. This provides an added protective benefit to situations when a patient needs to where the facemask 110 for periods of time that exceeds the recommended effective usage lifespan of the filter media 155.

The capnography facemask assembly embodiment 100 as described above can further comprise a front facemask retention brace 120 that is connected to the facemask 110 via a retention brace center connecting aperture 122 that encircles the facemask oxygen inlet tube 208. The front facemask retention brace 120 extending to brace distal ends 126 on either side of the facemask 110. A facemask retention strap 130 can connect to the facemask retention brace 120 essentially at the retention brace distal ends 126, as shown in FIGS. 1 and 3. The retention brace 120 and the retention strap 130 can help reduce that space in the facemask as discussed above. Certain embodiments envision the retention brace 120 pulling pressure on the facemask 110 in a location between a patient’s/wearer’s nostrils and mouth. In one optional embodiment, the filter media consists of a porosity that is less than 0.3 microns, which is at least an N-95 filter material.

Still another embodiment of the present invention contemplates a method for using capnography facemask assembly 100. The method comprising providing a facemask 110 that includes a face covering cup 201, a capnometer 160, and an exhaled air filter 150, wherein the face covering cup 201 terminates at a cup rim 212, which defines a cup exterior 205 and a cup interior 203. The capnometer 160 having a capnometer intake port 165 and capnometer exit port 166. An exhaled air filter 150 is interposed between the capnometer 160 and an exhale port 204 located at a cup mouth region 244 of the face covering cup 201. The next step envisions putting on a facemask 110 by covering a mouth, nostrils and a portion of a face of a person with the facemask 110 with the cup interior 203 facing the face, wherein the cup rim 212 essentially seals against the face. It should be noted that the volume between the cup interior 203 and the face defines an interior environment. The step for attaching the capnometer intake port 165 to a filter sample port 157 of the exhaled air filter 150 via a sample exhaled air tube 152 can occur before your after putting on the facemask 110. After the facemask 110 is strapped on the wearer’s face and attached to the capnometer 160, essentially all of the air that exits from the interior environment is filtered before entering an exterior environment 170 via the exhaled air filter 150. The filtering step produces filtered air 154, hence “(filtered air)”. Accordingly, all of the filtered air 154 exits into the exterior environment 170 via the capnometer exit port 166 and at least one primary exit aperture 159 of the exhaled air filter 150. In this way, essentially no contaminated air/breath flows into the exterior environment 170, which can endanger staff working around the person wearing the facemask 110.

The method embodiment further includes the exhaled air filter 150 being attached to the facemask 110 and covers the exhale port 204, as shown in FIG. 3.

The method embodiment as further shown in FIG. 4 includes the exhaled air filter 150 comprising filter media 155 that filters out viruses and bacteria from the air that exists in the interior environment, which is the space/volume between the wearer’s face in the facemask interior 203.

The method embodiment can further include wherein the facemask 110 also comprises a facemask oxygen inlet tube 208 located at a cup nose region 242 of the face covering cup 201. Hence, one can complete the step of connecting the facemask oxygen inlet tube 208 to an oxygen source 144 and flowing oxygen from the oxygen source 144 into the interior environment. In this way, the wearer has oxygen flowing towards their nostrils so they can breathe oxygenated air from the oxygen source 144.

The method embodiment further contemplates measuring carbon dioxide levels from a portion of the filtered air 154 at the capnometer 160. The portion of the filtered air 154 passes or otherwise flows from the filter sample port 157 through the sample exhaled air tube 152 into and through the capnometer 160, where the CO2 concentration of the filtered exhaled air 154 can be sampled (where it is displayed on the capnometer display 162) and out the capnometer exit port 166. In this way, even the sampled breath from the wearer/patient is filtered before it enters the exterior environment 170.

It is to be understood that even though numerous characteristics and advantages of various embodiments of the present invention have been set forth in the foregoing description, together with the details of the structure and function of various embodiments of the invention, this disclosure is illustrative only, and changes may be made in detail, especially in matters of structure and arrangement of parts within the principles of the present invention to the full extent indicated by the broad general meaning of the terms in which the appended claims are expressed. For example, different configurations, thicknesses, permeability, compressibility of the exhaled air filter 150 can be used without departing from the scope and spirit of the present invention. Also, though different facemask embodiments can be inventive as a whole, individual facemask components or elements can be equally inventive and stand alone. Further, the terms “one” is synonymous with “a”, which may be a first of a plurality.

It will be clear that the present invention is well adapted to attain the ends and advantages mentioned as well as those inherent therein. While presently preferred embodiments have been described for purposes of this disclosure, numerous changes may be made which readily suggest themselves to those skilled in the art and which are encompassed in the spirit of the invention disclosed.

What is claimed is:

1. A capnography facemask comprising:
a facemask cup possessing an outer rim defined by a nose bridge rim region configured to traverse a wearer’s nose bridge, two cheek rim regions configured to traverse a wearer’s cheeks, and a chin rim region configured to traverse under a wearer’s mouth,
the face covering cup defining a nose region configured to cover the wearer’s nose and a mouth region configured to cover the wearer’s mouth;
a facemask oxygen inlet tube outwardly extending from the facemask cup in the nose region;
the capnography facemask connected to an exhaled air filter located at the mouth region, the exhaled air filter comprising at least one primary exit aperture and a sample exit port,
the capnography facemask is configured to be worn on a wearer’s face when being used,
there is nothing between the at least one primary exit aperture and an exterior environment when the capnography facemask is being used,
there is a capnometer between the sample exit port in the exterior environment when the capnography facemask is being used.

2. The capnography facemask of claim 1, wherein the facemask oxygen inlet tube links to an oxygen source by way of an oxygen inlet tube.

3. The capnography facemask of claim 1, wherein the outer rim is configured to essentially seal against the wearer’s face when the capnography facemask is being used.

4. The capnography facemask of claim 1, wherein when the capnography facemask is being used, essentially all air configured to exit the capnography facemask is filtered air before entering the exterior environment.

5. The capnography facemask of claim 4, wherein a first portion of the filtered air is configured to exit into the exterior environment via the at least one primary exit aperture,
and a second portion of the filtered air is configured to exit into the exterior environment via a capnometer exit port of a capnometer, the capnometer connected to the sample exit port via a simple exhaled air tube.

6. The capnography facemask of claim 5, wherein the first portion is greater than the second portion.

7. The capnography facemask of claim 1, wherein the at least one primary exit aperture is a slotted aperture.

8. The capnography facemask of claim 1, further comprising a front facemask retention brace that defines a retention brace center located between the nose region and the mouth region,
the front facemask retention brace extends to retention brace distal ends on either side of the retention brace center toward the cheek rim regions.

9. The capnography facemask of claim 8, wherein a facemask retention strap connects to the facemask retention brace essentially at the retention brace distal ends.

10. The capnography facemask of claim 8, wherein the front facemask retention brace comprises a brace aperture at the retention brace center,
the brace aperture and circles the facemask oxygen inlet tube.

11. A capnography facemask assembly comprising:
a facemask including: 1) a face covering cup defining a cup exterior and a cup interior, the face covering cup terminates at a cup rim, 2) the facemask further comprising a facemask oxygen inlet tube located at a cup nose region and extending outwardly from the cup exterior, and 3) an exhale port located at a cup mouth region,
the facemask configured to cover a mouth and nostrils of a person and essentially seal against a face of a person at an interface between the face and the cup rim, the cup interior facing the face and the cup exterior facing an exterior environment when worn on the face;
a capnometer comprising a capnometer intake port and a capnometer exit port;
an exhaled air filter disposed between the capnometer and the exhale port,
the exhaled air filter comprising filter media, at least one primary exit aperture, and a sample exit port, wherein the exterior environment is in communication with the cup interior via the at least one primary exit aperture and via the capnometer exit port.

12. The capnography facemask assembly of claim 11 further comprising an oxygen source connected to the facemask oxygen inlet tube, the exterior environment is only in communication with the cup interior via the at least one primary exit aperture and via the capnometer exit port.

13. The capnography facemask assembly of claim 11, wherein the exhaled air filter is connected to the facemask and covers the exhale port.

14. The capnography facemask assembly of claim 11 further comprising a front facemask retention brace that is connected to the facemask via a retention brace center connecting aperture that encircles the facemask oxygen inlet tube,
the front facemask retention brace extending to brace distal ends on either side of the facemask.

15. The capnography facemask assembly of claim 11, wherein the filter media consists of a porosity that is less than 0.3 microns.

16. A method for using a capnography facemask assembly, the method comprising:
providing a facemask that includes a face covering cup, a capnometer, and an exhaled air filter, wherein the face covering cup terminates at a cup rim, which defines a cup exterior and a cup interior,
the capnometer having a capnometer intake port and capnometer exit port, and an exhaled air filter interposed between the capnometer and an exhale port located at a
cup mouth region of the face covering cup;
covering a mouth, nostrils and a portion of a face of a person with the facemask with the cup interior facing the face, wherein the cup rim essentially seals against the face, volume between the cup interior and the face defines an interior environment;
attaching the capnometer intake port to a filter sample port of the exhaled air filter via a sample exhaled air tube;
filtering essentially all air that exits from the interior environment to an exterior environment via the exhaled air filter (filtered air),
all the filtered air exits into the exterior environment via the capnometer exit port and at least one primary exit aperture of the exhaled air filter.

17. The method of claim 16, wherein the exhaled air filter is attached to the facemask and covers the exhale port.

18. The method of claim 16, wherein the exhaled air filter comprises filter media that filters out viruses and bacteria from the air that exists in the interior environment.

19. The method of claim 16, wherein the facemask further comprises a facemask oxygen inlet tube located at a cup nose region of the face covering cup, connecting the facemask oxygen inlet tube to an oxygen source and flowing oxygen from the oxygen source into the interior environment.

20. The method of claim 16 further comprising measuring carbon dioxide levels from a portion of the filtered air at the capnometer, the portion of the filtered air passes from the filter sample port through the sample exhaled air tube through the capnometer and out the capnometer exit port.
